(19) United States
(12) Patent Application Publication
(10) Pub. No.: US 2005/0021064 A1
(43) Pub. Date: Jan. 27, 2005

(54) Disposable surgical scalpel

(76) Inventor: Sang-Ho Lee, Sasang-gu (KR)

Correspondence Address: BIRCH STEWART KOLASCH & BIRCH, PO BOX 747, FALLS CHURCH, VA, 22040-0747, US

(21) Appl. No.: 10/700,551

(22) Filed: Nov. 5, 2003

(30) Foreign Application Priority Data
Jul. 23, 2003 (KR) 2003-0050686

(51) Int. Cl. A61B 17/32

(52) U.S. Cl. 606/167

(57) ABSTRACT

A disposable surgical scalpel includes a sharp blade for cutting skin or body parts in a surgical operation, a blade-fixing member to which a rear end of the sharp blade is integrally fixed such that the sharp blade can reciprocate together with the blade-fixing member, and a handle disposed enclosing the blade-fixing member such that the sharp blade fixed on the blade-fixing member can be completely projected out of and inserted into the handle.

### BACKGROUND OF THE INVENTION

1. Field of the Invention

The present invention relates to a disposable surgical scalpel assembly, and more particularly, to a surgical scalpel assembly that is designed to be convenient in use and to preemptively prevent an accident caused by the surgical scalpel.

2. Background of the Prior Art

Generally, a surgical scalpel is an instrument used for cutting skin or body parts in a surgical operation. Such a surgical scalpel is formed in a variety of shapes according to its use and purpose.

FIG. 1 shows a conventional surgical scalpel.

As shown in the drawing, a conventional surgical scalpel comprises a sharp blade 10 and a handle 11 to which a rear end of the sharp blade 10 is attached. To perform a surgical operation, a surgeon grasps the handle 11 and cuts an affected part with the sharp blade 10 contacting a part to be operated upon.

When the surgical scalpel is cleaned and reused, there may be a danger of infectious contamination. Therefore, disposable surgical knives are widely used.

However, when a disposable surgical scalpel that is used for the surgical operation is disposed of, due to the sharp blade, persons may be injured or other articles may be damaged.

Even before a surgical operation, since the sharp blade 10 is externally exposed, a surgeon may be injured by carelessness when grasping the surgical scalpel. Furthermore, when the sharp blade 10 contacts other articles, the blade may be damaged.

Accordingly, as shown in FIG. 1, a protecting cap 12 is provided to cover the sharp blade 10. The protecting cap 12 is fitted on the surgical scalpel such that it can enclose the sharp blade 10 to protect the same.

That is, the protecting cap 12 prevents the sharp blade 10 from contacting other articles, thereby preventing the sharp blade 10 from being blunted or damaged. Further, the protecting cap 12 can prevent the sharp blade 10 from directly contacting a human body or other articles, thereby preventing the persons or articles from being injured or damaged.

Namely, the surgical scalpel is normally stored in a state where the protecting cap 12 is fitted thereon to enclose the sharp blade 10. When in use for the surgical operation, the protecting cap 12 is separated from the sharp blade 10 of the surgical scalpel.

When the surgical operation is finished, the surgical scalpel is disposed in a state where the protecting cap 12 is fitted to cover the blade 10.

The separation and fitting of the protecting cap requires the user to use both hands. That is, by using only one hand, it is impossible to separate or fit the cap from or on the surgical scalpel.

Accordingly, it is inconvenient to use both hands always to separate or fit the protecting cap from or on the surgical scalpel. Furthermore, in the course of separation or fitting of the protecting cap from or on the surgical scalpel, the user may be injured by the blade due to his/her carelessness, whereby the user may be cross infected.

### SUMMARY OF THE INVENTION

Therefore, the present invention has been made in an effort to solve the above-described problems of the conventional arts.

An object of the present invention is to provide a disposable surgical scalpel that is convenient to use by being designed to be handled using only one hand.

Another object of the present invention is to provide a disposable surgical scalpel that can prevent the careless accident in advance as well as the damage of the sharp blade by enclosing the blade using a protecting cover.

To achieve these objects and other advantages and in accordance with the purpose of the invention, as embodied and broadly described herein, there is provided a disposable surgical scalpel comprising a sharp blade for cutting skin or body parts in a surgical operation; a blade-fixing member to which a rear end of the sharp blade is integrally fixed such that the sharp blade can be reciprocated together with the blade-fixing member; and a handle disposed enclosing the blade-fixing member such that the sharp blade fixed on the blade-fixing member can be completely projected out of and inserted into the handle.

It is to be understood that both the foregoing general description and the following detailed description of the present invention are exemplary and explanatory and are intended to provide further explanation of the present invention as claimed.

### BRIEF DESCRIPTION OF THE DRAWINGS

The accompanying drawings, which are included to provide a further understanding of the present invention and are incorporated in and constitute a part of this application, illustrate embodiment(s) of the present invention and together with the description serve to explain the principle of the present invention. In the drawings:

FIG. 1 is a perspective view of a conventional disposable surgical scalpel;

FIG. 2 is a perspective view of a disposable surgical scalpel according to an embodiment of the present invention;

FIG. 3 is an exploded perspective view of a disposable surgical scalpel according to an embodiment of the present invention;

FIG. 4a is a sectional view of a disposable surgical scalpel according to an embodiment of the present invention, in which a sharp blade is inserted in a handle; and

FIG. 4b is a sectional view of a disposable surgical scalpel according to an embodiment of the present invention, in which a sharp blade is projected out of a handle.

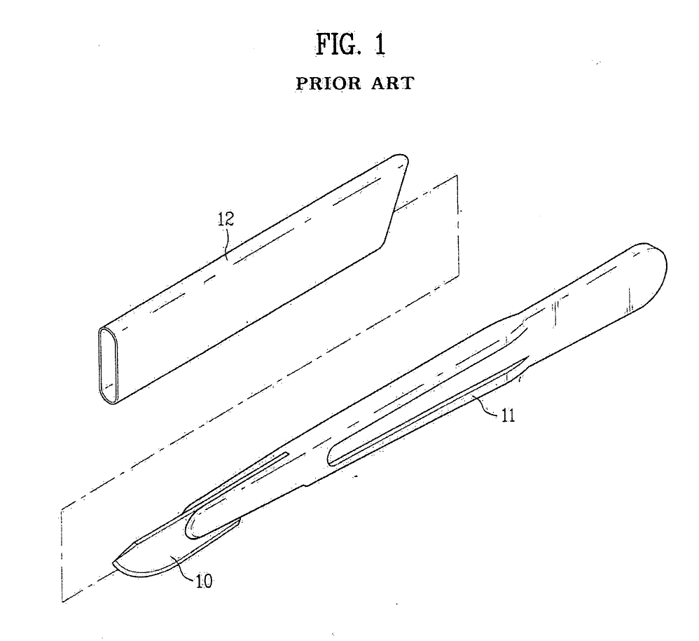

### DETAILED DESCRIPTION OF THE PREFERRED EMBODIMENTS

Reference will now be made in detail to a preferred embodiment of the present invention with reference to the accompanying drawings.

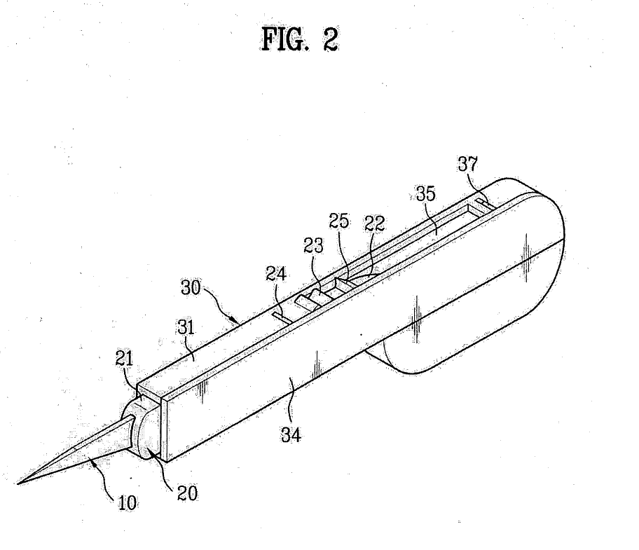

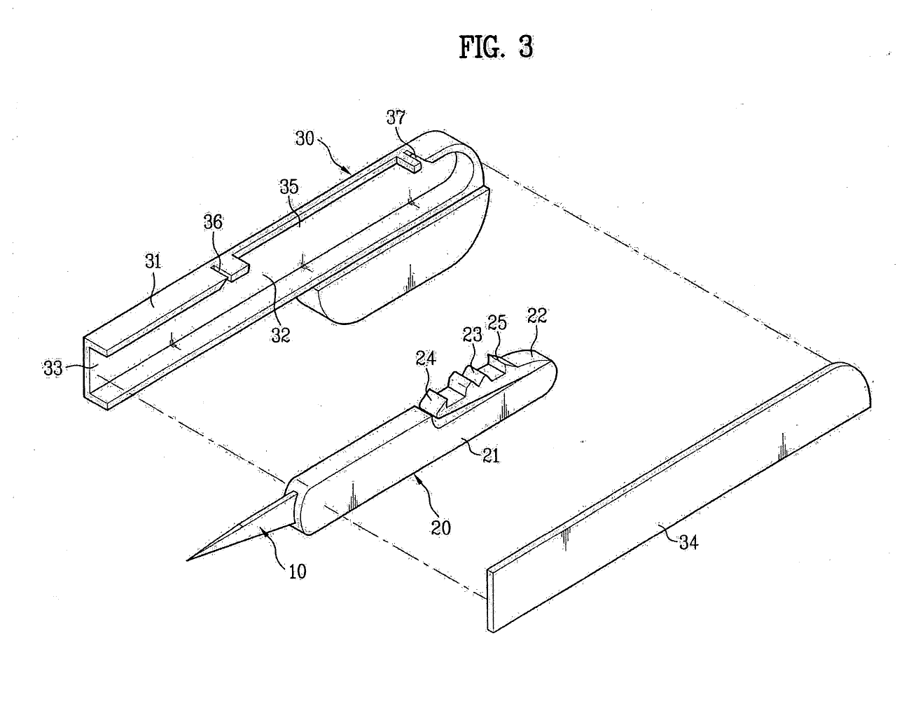

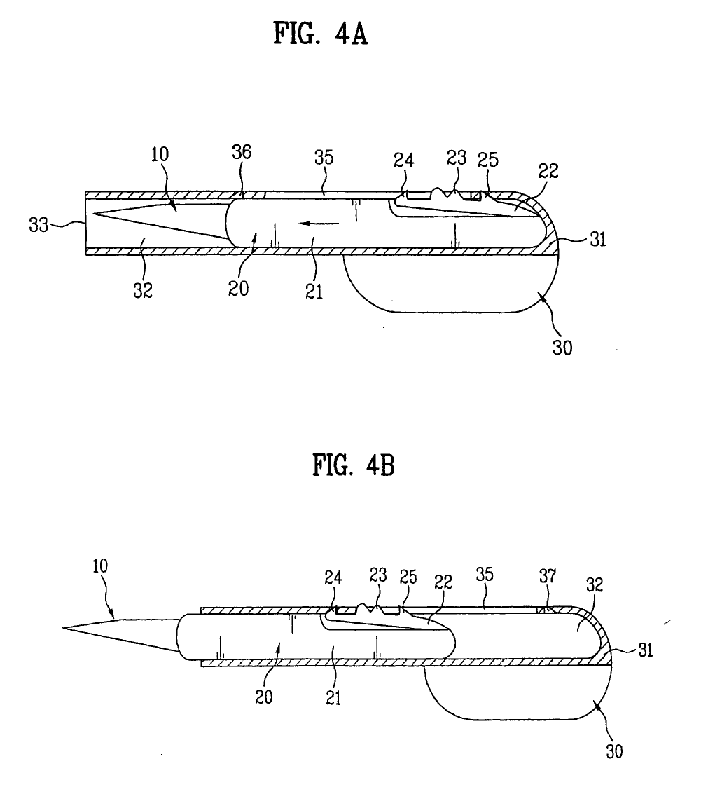

FIG. 2 is a perspective view of a disposable surgical scalpel according to an embodiment of the present invention, FIG. 3 is an exploded perspective view of a disposable surgical scalpel according to an embodiment of the present invention, FIG. 4a is a sectional view of a disposable surgical scalpel according to an embodiment of the present invention, in which a sharp blade is inserted in a handle, and FIG. 4b is a sectional view of a disposable surgical scalpel according to an embodiment of the present invention, in which a sharp blade is projected out of a handle.

As shown in the drawings, the disposable surgical scalpel according to the present invention comprises a sharp blade 10 for cutting skin or body parts in a surgical operation, a blade-fixing member 20 to which a rear end of the sharp blade 10 is integrally fixed such that the sharp blade 10 can be reciprocated together with the blade-fixing member 20.

A handle 30 is disposed enclosing the sharp blade 10 and the blade-fixing member 20. The user grasps the handle in the surgical operation. A front end of the handle 30 is designed such that the sharp blade 10 can be inserted into or projected out of the handle 30 through thereof by the reciprocation motion of the blade-fixing member 20.

As shown in FIG. 3, the blade-fixing member 20 includes a body for preventing the blade 10 from moving in the course of the surgical operation. That is, the blade-fixing member is securely fixed on the body 21.

A reciprocal motion aid member 22 is coupled on a top surface rear end of the body 21. The reciprocal motion aid member 22 has a free front end and a rear fixed end by which the free front end is elastically biased upwards.

That is, the blade-fixing member 20 is designed to reciprocate in a state where the free front end of the reciprocal motion aid member 22 is depressed by outer force and to maintain its moved position when the outer force is released to return the free front end of the reciprocal motion aid member 22 to its initial upward position.

The reciprocal motion aid member 22 is provided at a top surface thereof with a pushing portion 23. That is, the user reciprocates the blade-fixing member in the handle 30 in a state where his/her finger contacts the pushing portion 23. A plurality of grooves or a comb structure is formed on the pushing portion 23 so as to prevent the finger contacting the pushing portion 23 from being slipped.

The reciprocal motion aid member 22 is further provided at the free front end with a first fixing projection 24 protruded upwards for fixing the reciprocal motion aid member 22 to prevent the blade-fixing member 20 from moving when the sharp blade 10 is projected out of the handle 30 by the forward movement of the blade-fixing member 20.

The reciprocal motion aid member 22 is further provided at a rear side of the pushing portion 23 with a second fixing projection 25 protruded upwards for fixing the reciprocal motion aid member 22 to prevent the blade-fixing member 20 from being projected out of the handle 30 moving when the sharp blade 10 is inserted into the handle 30 by the rearward movement of the blade-fixing member 20.

That is, when the surgical scalpel is not being used, the sharp blade 10 is prevented from being projected out of the handle 30 by fixing the blade-fixing member 20 using the second fixing projection 25. When the surgical scalpel is being used for the surgical operation, the sharp blade 10 projected out of the handle 30 is prevented from being pushed into the handle 30 by fixing the blade-fixing member 20 using the first fixing projection 24.

The first and second fixing projections 24 and 25 are slightly inclined toward the pushing portion 23 to securely fix the blade-fixing member 20.

Meanwhile, as shown in FIG. 3, the handle 30 comprises a main body 31 designed to enclose the blade-fixing member 20. An outer shape of the main body 31 can be designed in consideration of a length and shape of the sharp blade 10 or the user's grasping convenience.

The main body 31 of the handle 30 is provided with a cavity 32 in which the blade-fixing member 20 to which the blade 10 is fixed is reciprocally disposed. The main body 31 is further provided at a front end thereof with a blade passing opening 33 through which the blade 10 can be projected out of or inserted in the handle 30.

In addition, a separation-preventing plate 34 is coupled on a main body 31 to close an opened side portion of the main body, thereby preventing the blade-fixing member 20 from being separated from the cavity 32.

A reciprocal motion aid slot 35 through which the pushing portion 23 of the reciprocal motion aid member 22 is projected is formed on a top surface of the main body 31 of the handle 30. A length of the reciprocal motion aid slot 35 is designed to limit the reciprocal motion of the blade-fixing member 20 to an extent identical to a maximum projected length of the blade 10 out of the handle 30.

A first fixing groove 36 is formed on the top surface of the main body 31 at a front side of the reciprocal motion aid slot 25. That is, when the blade 10 is projected out of the handle 30, the first fixing projection 24 of the blade-fixing member 20 is interlocked with the first fixing groove 36, thereby preventing the blade 10 from moving during the surgical operation.

In this embodiment, although only one fixing groove is formed, plural grooves may be formed to be capable of adjusting the projected length of the blade out of the handle 30. A second fixing groove 37 is formed on the top surface of the main body 31 at a rear side of the reciprocal motion aid slot 25. That is, when the blade 10 is inserted into the handle 30, the second fixing projection 25 of the blade-fixing member 20 is interlocked with the second fixing groove 37, thereby preventing the blade 10 from being projected out of the hand when the surgical scalpel is not being used.

In addition, to prevent the blade-fixing member 20 disposed in the cavity 32 of the handle 30 from swaying or moving, the blade-fixing member 20 is disposed tightly contacting an inner surface of the cavity 32.

The operation of the above-described surgical scalpel will be described hereinafter.

From an initial portion where the blade 10 is inserted into the handle 30, when the user applies force to the pushing portion 23 of the reciprocal motion aid member 22, which is projected through the reciprocal motion aid slot 35 formed on the top surface of the handle 30, the free front end of the reciprocal motion aid member 22 is depressed toward the blade-fixing member 20.

At this point, the second fixing projection 25 formed on the pushing portion 23 of the reciprocal motion aid member 22 and interlocked with the second fixing groove 37 formed on the main body 31 of the handle 30 is released from the second fixing groove 37.

Therefore, the whole body of the reciprocal motion aid member 22 is to be located in the cavity 35.

Afterwards, when the pushing portion 23 of the reciprocal motion aid member 22 is pushed in an arrow direction depicted in FIG. 4a, the blade-fixing member 20 moves forwards along the cavity 32.

That is, as the blade-fixing member 20 moves forwards in the handle 30, the blade 10 fixed on the blade-fixing member 20 is projected out of the handle 30 through the blade passing opening 33 formed on the front end of the handle 30.

When the blade 10 is projected out of the handle to a predetermined extent, the user release the force applied to the reciprocal motion aid member 22 so that the reciprocal motion aid member 22 can be returned to the initial location due to the self-elastic force.

At this point, the first fixing member 24 formed on the free front end of the reciprocal motion aid member 22 is interlocked with the first fixing groove 36 formed on the top surface of the handle 30, thereby securely fixing the blade-fixing member 20 to thereby securely fix the blade 10.

In this state, the surgical scalpel is used for the surgical operation.

When the surgical operation is finished and the user applies force to the pushing portion 23 of the reciprocal motion aid member 22, which is projected through the reciprocal motion aid slot 35 formed on the top surface of the handle 30, the first fixing projection 24 is released from the first fixing groove 36.

Afterwards, the user pushes the pushing portion 23 rearwards to move the blade-fixing member 20 rearwards along the cavity 32 of the handle 30 until the blade 10 is completely inserted into the handle 30 as shown in FIG. 4.

Next, when the user release the force applied to the reciprocal motion aid member 22 so that the reciprocal motion aid member 22 can be returned to the initial location due to the self-elastic force.

At this point, the second fixing member 245 of the reciprocal motion aid member 22 is interlocked with the second fixing groove 37 formed on the top surface of the handle 30, thereby securely fixing the blade-fixing member 20 to thereby securely fix the blade 10 in the handle.

In this state, the surgical scalpel is disposed.

As described above, the disposable surgical scalpel of the present invention has an advantage in that it is designed to project and insert the blade out of and in the handle by manipulating the reciprocal motion aid member of the blade-fixing member using only one hand, thereby providing the convenience in use to the user.

The disposable surgical scalpel of the present invention has a further advantage in that it can prevent the damage of the sharp blade by enclosing the blade using a protecting cover. The disposable surgical scalpel of the present invention has a further advantage in that it can prevent the careless accident in advance by preventing the blade from contacting the human body.

The foregoing embodiment is merely exemplary and is not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

1. A disposable surgical scalpel comprising:
a sharp blade for cutting skin or body parts in a surgical operation;
a blade-fixing member to which a rear end of the sharp blade is integrally fixed such that the sharp blade can reciprocate together with the blade-fixing member; and
a handle disposed enclosing the blade-fixing member such that the sharp blade fixed on the blade-fixing member can be completely projected out of and inserted into the handle.

2. The disposable surgical scalpel of claim 1, wherein the blade-fixing member comprises:
a body to which the sharp blade is integrally coupled;
a reciprocal motion aid member coupled on a top rear end of the body, the reciprocal motion aid member having a free front end and a rear fixed end by which the free front end is elastically biased upwards; and
a pushing portion protruded from a top surface of the reciprocal motion aid member, a user's finger contacting the pushing portion to reciprocate the blade-fixing member;
a first fixing projection protruded from the top surface of the reciprocal motion aid member at a front side of the pushing portion to fix the blade-fixing member at a location where the blade is projected out of the handle; and
a second fixing projection protruded from the top surface of the reciprocal motion aid member at a rear side of the pushing portion to fix the blade-fixing member at a location where the blade is inserted into the handle.

3. The disposable surgical scalpel of claim 2, wherein the handle comprising:
a main body defining a cavity for receiving the blade-fixing member, the main body being provided at a front end with a blade passing opening through which the sharp blade can be projected out and inserted in;
a separation-preventing plate fixed on a side portion of the main body to prevent the blade-fixing member receiving in the cavity from being separated;
a reciprocating motion aid slot formed on a top surface of the main body to obtain a moving distance of the pushing portion of the reciprocating motion aid member and to thereby allow the blade-fixing member to move at a predetermined distance;
a first fixing groove with which the first fixing projection is interlocked when the sharp blade is projected out of the handle by a forward movement of the blade-fixing member, the first fixing groove being formed on a front side of the reciprocating motion aid slot; and
a second fixing groove with which the second fixing projection is interlocked when the sharp blade is inserted in the handle by a rearward movement of the blade-fixing member, the first fixing groove being formed on a rear side of the reciprocating motion aid slot.
